(19) United States
(12) Patent Application Publication
(10) Pub. No.: US 2008/0306385 A1
(43) Pub. Date: Dec. 11, 2008

(54) Automatic Ultrasound Scanning Initiated by Protocol Stage

(75) Inventors: James Jago, Seattle, WA (US); Martin Anderson, Bothell, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/097,826

(22) PCT Filed: Dec. 14, 2006
(86) PCT No.: PCT/IB2006/054864

§ 371 Date: Jun. 17, 2008

Related U.S. Application Data
(60) Provisional application No. 60/751,537, filed on Dec. 19, 2005.

(51) Int. Cl. A61B 8/00

(52) U.S. Cl. 600/443

(57) ABSTRACT

The invention relates to a method and system for automatically initiating user control automation by one of either beginning a Protocol stage for imaging or changing dependent controls. This invention eliminates the need for user intervention for hitting a button such as the I-Scan button for the Philips I-Scan system, which is one of the systems on which the invention can be implemented.

The present invention relates to an ultrasound scan that attempts to automatically optimize gain, time gain compensation (TGC) and compression in order to present an optimum image for any subject or view. In particular, the present invention relates to the automatic adjustment of user controls for a two dimensional scan being automatically initiated at (a) the beginning of each stage in a protocol or (b) after a specific manual control change such an automatic adjustment can be an algorithmic adjustment. One type of auto-opt or user control adjustments for a two dimensional scan is I-Scan (by Philips Medical Systems). Protocols are a feature that involve stepping the user through a sequence of standard imaging stages that could include particular anatomical views, imaging modes, analysis stages, etc. This concept is well established in cardiology and is beginning to be adopted in some gastrointestinal (GI) applications.

The present invention relates to a method and system for automatically initiating automated user control adjustments for ultrasound imaging equipment. The quality of an ultrasound image can be affected by many variables—including operator experience, system capabilities, and patient variability. In an attempt to deal with this variability in image quality, many ultrasound systems—especially High-end and Premium ultrasound systems designed to have optimum image quality—typically have a large number of user-adjustable controls available to optimize the imaging performance for any particular patient or organ. These include, for 2D grayscale imaging, controls for overall gain, time gain compensation (TGC), lateral gain compensation (LGC), compression, imaging frequency, focus depth, imaging depth, frame-rate, and many others.

Unfortunately this approach has several disadvantages, including:

tendency to increase scan times (because of the need to adjust the imaging controls frequently) and hence reduce department efficiency

users must be trained on and understand how the controls work

adds complexity to user interfaces and hence tends to limit minimum system size.

In an attempt to deal with this control complexity, many ultrasound manufacturers have developed automated or semi-automated means to adjust some of these controls separately or in groups—for example I-Scan (Philips Medical Systems) automatically sets gain, TGC, LGC, and compression, Tissue Equalization Technology or TEQ (Siemens) automatically sets gain, TGC, and LGC, Automatic B-mode Optimization or ABO (GE) automatically sets gray map. These features improve user workflow by reducing the number of times the user must manually adjust the relevant controls within a given exam.

The present invention provides a method and a system for automatically initiating user control automation at the beginning of each Protocol stage. In addition the present invention provides for a method and a system that automatically launches user control automation system such as by way of illustrative example Philips I-Scan when the user manually adjusts a control that might invalidate the I-Scan analysis.

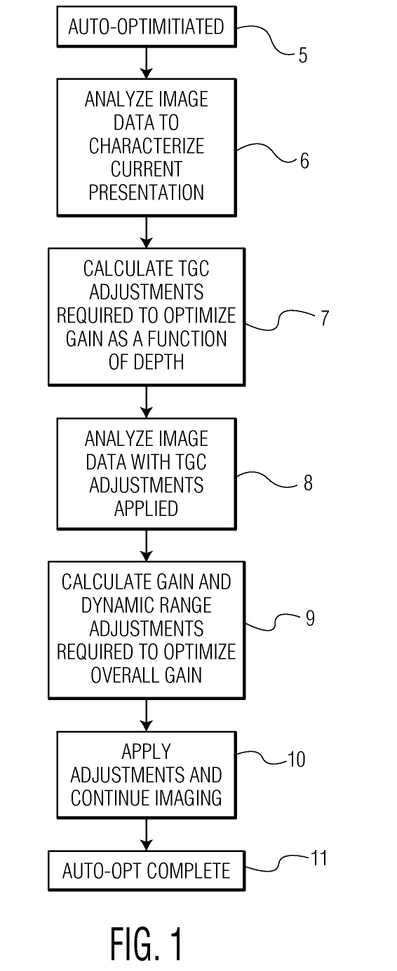

FIG. 1 is a flow chart illustrating the known auto-opt algorithm's processing steps;

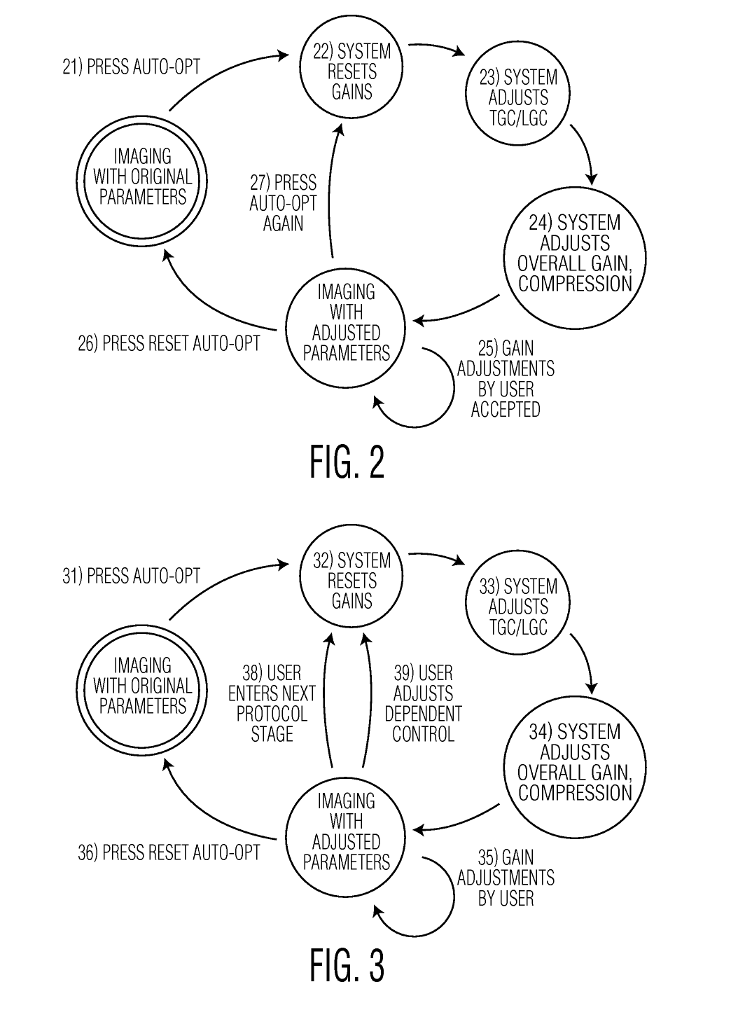

FIG. 2 illustrates the known operation of auto-opt from a user's perspective; and

FIG. 3 illustrates the present invention showing the operation of auto-opt with more automated initiation of the workflow.

Referring now to the drawings, FIG. 1 shows one type of user control automation or auto-opt although it is understood that the present invention can be adapted to work on any ultrasound imaging system. FIG. 1 for example, shows the steps the system takes each time the auto-opt button is pressed. These algorithms can also help to improve imaging consistency by reducing the dependence on a skilled operator.

Two dimensional scanning with ultrasound equipment is known and used for medical applications such as Cardiology and GI. I-Scan automated systems includes HDI 5000, HD11, iU22, and iE33—all Philips ultrasound equipment incorporates I-Scan capability.

Two limitations of I-Scan, and of many of the automated features provided by other Ultrasound Manufacturers, are (1) it requires the user to press a button to activate it, and (2) it does not always perform reliably. It would be desirable to launch I-Scan automatically, and thereby improve workflow. It would further be desirable to automatically initiate I-Scan at the beginning of each stage in the Protocol so that the additional context provided by the Protocol stage may also be used to improve the reliability of I-Scan itself.

FIG. 1 shows the auto-opt processing steps of the algorithm each time the auto-opt button is pressed. These steps include: initiating auto-opt (5), analyzing image data to characterize current presentation, calculating TGC (time gain control) adjustments required to optimize gain as a function of depth (6), analyzing image data with TGC adjustments applied (7), calculating Gain and Dynamic Range adjustments required to optimize overall gain (8), applying adjustments and continuing imaging (9), and auto-opt completed (10).

The auto-opt system is software implemented in ultrasound equipment. For example, the I-scan system is software implemented in Philips ultrasound equipment such as HDI 5000; HD11; iU22; and iE33 equipment.

Although the existing approaches to control automation are very useful features, they each suffer from some limitations—especially with respect to how the user interacts with them. This can be illustrated as shown in FIG. 2. For example, consider the I-Scan feature—the basic operation of which is shown in FIG. 2, there is still a considerable amount of user interaction:

The user must hit a button to initiate auto-opt (step 21), which then causes the system to analyze the image and select new settings for the relevant controls.

Since the auto-opt analysis only applies to the target being viewed when the auto-opt button is pressed, if the view has changed (e.g. the user has moved the transducer) or the user has manipulated a control(s) that affects the need to make automated control adjustments (steps 25 or 26), the user may need to re-initiate auto-opt (step 27).

An alternative solution to this problem is to have the automation algorithm run continuously, thus eliminating the need for repeated user initiation on scene or other control changes. One example of this type of automation is Native TEQ (Siemens), which operates pseudo-continuously based on detecting scene changes or based on a timer. U.S. Pat. No. 6,542,626 by GE triggers re-optimization based on image brightness histogram changes. One disadvantage of continuous or pseudo-continuous operation is that many of the controls being automated either interrupt the signal path, possibly causing the image to “flicker” or the cineloop to be lost. Another limitation of this approach is that the analysis and correction must be applied very quickly—which is not possible with some controls on some systems—to avoid continuously interrupting the user's imaging. Another limitation is that the system may trigger re-optimization at a very inopportune time, such as when the clinician is about to print or save an image after adjusting the controls to their preferences.

Another limitation of existing automation algorithms is their reliability—ie. how consistently they apply control adjustments that an expert user would have made manually. This requirement is particularly important for continuous automation, since the controls are being updated frequently. Since the algorithm is essentially trying to predict the control changes that a human user would make, the algorithm must work with as much information as possible about the image being analyzed. Existing algorithms extract various properties of the image—such as its gray scale statistics, amplitude trends, noise segmentation, etc—but, unlike a human operator, they know little or nothing about the context of the image, ie. they do not know what kind of target (e.g. organ type, pathology, location within the organ). Having knowledge of this kind of contextual information is likely to significantly improve the reliability of the automated control adjustments.

Thus, the present invention provides for improving automation control and is designed to address the shortcomings of the aforementioned existing automation algorithms.

FIG. 3 illustrates the operation of the present invention showing how the present invention overcomes some of the limitations of the existing auto-opt feature by the following modifications that are not mutually exclusive:

Automatically launching auto-opt at the beginning of a Protocol stage.

Automatically launching auto-opt when the user adjusts a control that might invalidate the auto-opt analysis—for example imaging depth, imaging frequency, etc.—ie. an auto-opt dependent control.

The present invention can be implemented as a simple modification to existing automation algorithms (such as auto-opt) by replacing a manual auto-opt initiation (step 7 in FIG. 2 with automatic auto-opt initiation driven by either Protocol Stage (step 8 in FIG. 3) or by another user control (step 9 in FIG. 3).

Steps 31-36 are the same as the known auto-opt algorithm shown in FIG. 2. (steps 21-26) The auto-opt system detects if either one of the two following conditions occurs. Step 28 the user enters the next Protocol stage or step 29 the user adjusts a dependent control. When one of these two steps 28 or 29 are detected by software code, the system automatically activates the auto-opt button without the need for user intervention.

The present invention can initiate auto-opt automatically when entering a Protocol stage. Protocols are a relatively recent addition to ultrasound systems that attempt to improve workflow, and assist the user, by prompting the user through various stages of the current exam and providing tools and settings appropriate to that stage. For example, a Cerebro-Vascular protocol might prompt the user to go through the following stages:

Acquire a Common Carotid Artery (CCA) image and PW waveforms

Acquire an External Carotid Artery (ECA) image and PW waveforms

Acquire an Internal Carotid Artery (ICA) image and PW waveforms

At each stage, appropriate systems settings (e.g. depth, PW sample volume position, etc) are automatically set and appropriate tools (e.g. measurements, annotations) are provided. The present invention adds auto-opt initiation to that list, since the imaging view and context will have changed for each stage. This will largely eliminate the need for the user to manually initiate auto-opt, but without the limitations of continuous auto-opt since imaging will be interrupted anyway by moving the transducer to the next clinical target. (This benefit assumes that the transducer has been repositioned on the next target before the next stage of the protocol is selected. This is merely a user training issue.)

This approach also allows the reliability and consistency of the auto-opt algorithm to be improved by taking account of the information provided by the Protocol stage. For example, in a Cardiac protocol the internal system parameters used to optimize auto-opt would be set differently between apical and para-sternal views. Other examples exist for GI Protocols, such as might be used for OB—e.g. different auto-opt optimization for fetal head, fetal abdomen, femur length measurements, etc. Having more context (ie. from a Protocol stage) could also be used to drive image segmentation algorithms, which would give even more explicit information about the type and location of the target of interest and hence improve even more the reliability of auto-opt. For example, at the fetal head stage of an OB Protocol the algorithm could know to segment out the head itself from the rest of the image, and the automation algorithm would then analyze the head to determine optimum control settings.

In the future auto-opt is likely to include additional user controls such as imaging frequency selection, imaging depth, focus position, res-speed selection, etc. These additional elements are likely to be even more dependent on the contextual information provided by a protocol stage. It is also possible that the contextual information provided by a protocol could also be used to improve Doppler or, in the future, Color auto-opt—one example would be to use the context of the imaging anatomy to assist the automatic placement of a PW sample volume.

The present invention can also automatically initiate auto-opt when one of the dependent controls are changed (as shown in step 39 of FIG. 3). Even after auto-opt has been automatically initiated at the beginning of a new Protocol stage, there are still times when the user needs to adjust another control that is not set automatically by auto-opt and hence may make the auto-opt derived settings inappropriate. In this case, auto-opt can be initiated automatically whenever one of these controls are changed, thus re-adjusting the auto-opt automated controls without further user interaction. Again, this is an improvement on continuous auto-opt since many of these other controls (e.g. imaging frequency, depth, etc.) are already interruptive and already cause some delay in the signal path, so adding an auto-opt step should not be a problem.

User control automation algorithms or auto-opt algorithms such as Philips I-Scan and the modifications of the present invention described herein can be applied to any ultrasound imaging system and therefore the present invention is not limited to any one ultrasound imaging system such as the Philips' I-Scan System.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. A method of automatically initiating automatic user control adjustment for ultrasound imaging with ultrasound equipment, the steps comprising:
at least one of either entering a protocol stage for imaging or changing a dependent control; and
automatically initiating automatic user control adjustment for ultrasound imaging with ultrasound equipment in response thereto.

2. The method according to claim 1 wherein said automatic user control adjustment is automatically initiated by software.

3. The method according to claim 1 wherein said dependent control is imaging depth,

4. The method according to claim 1 wherein said dependent control is imaging frequency.

5. The method according to claim 1 wherein said automatic user control adjustment is Philips' I-Scan System.

6. A system of automatically initiating automatic user control adjustment for ultrasound imaging with ultrasound equipment comprising:
at least one of either a protocol stage of an imaging system being entered during imaging by an ultrasound equipment using automatic user control adjustment for ultrasound imaging or a dependent control of said ultrasound equipment being varied that drives an automatic initiation of said automatic user control adjustment.

7. The system according to claim 6 wherein software controls said automatic initiation of said automatic user control adjustment.

8. The system according to claim 6 wherein said dependent control is for imaging depth.

9. The system according to claim 6 wherein said dependent control is for imaging frequency.

10. The system according to claim 6 wherein said automatic user control adjustment is Philips' I-Scan System.
